(19) United States
(12) Patent Application Publication
(10) Pub. No.: US 2026/0363569 A1
(43) Pub. Date: Nov. 19, 2026

(54) HEART AND PULSE MONITORING

(71) Applicants: Richy YUN, Richmond, CA (US); David TAYLOR, Berkeley, CA (US)

(72) Inventors: Richy YUN, Richmond, CA (US); David TAYLOR, Berkeley, CA (US)

(21) Appl. No.: 19/681,076

(22) Filed: May 18, 2026

Related U.S. Application Data
(60) Provisional application No. 63/807,056, filed on May 16, 2025.

(51) Int. Cl. A61B 5/05; A61B 5/00; A61B 5/021; A61B 5/11

(52) U.S. Cl. CPC A61B 5/05; A61B 5/02125; A61B 5/02141; A61B 5/11; A61B 5/681; A61B 5/7221; A61B 2562/0223; A61B 2562/043

(57) ABSTRACT

A wearable device incorporating an array of acoustically driven ferromagnetic resonance (ADFMR) sensors measures biomagnetic signals associated with blood flow at multiple anatomical locations. The device separates magnetoplethysmographic and magnetocardiographic signals using frequency-domain filtering and computes pulse transit time from temporally aligned features. The pulse transit time is used to estimate blood pressure without requiring optical or electrical sensing, multi-limb instrumentation, or continuous calibration.

### CLAIM OF PRIORITY

This patent application claims priority to U.S. Provisional Patent Application No. 63/807,056, titled “HEART AND PULSE MONITORING,” and filed on May 16, 2025, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This invention relates generally to the field of magnetic sensors, particularly magnetic sensors based on ferromagnetic resonance; and more specifically to a new and useful system and method for a magnetic sensor array circuit.

### BACKGROUND

Heart and blood pressure monitoring is a crucial aspect of healthcare, particularly for patients with hypertension, cardiovascular disease, and other conditions that may require periodic monitoring or blood pressure. Traditional blood pressure monitoring methods and devices are bulky and can be difficult for a casual user, such as a typical patient, to operate.

Recent advancements in technology have led to the development of patient-based blood pressure monitoring systems that can be worn on the body. These systems typically use photoplethysmography (PPG) or electrocardiography (ECG) sensors. Systems employing PPG or ECG sensors may lack sensitivity and be subject to interference from clothing, adequate skin contact and the like.

What is needed is a heart and blood pressure measuring device to provide accurate and reliable measurement without complicated calibration procedures or specialized training.

### SUMMARY OF THE DISCLOSURE

Described herein are apparatuses, systems, and methods to detect and measure one or more metrics (e.g., waveforms) indicating a patient's physiological status, including but not limited to one or more of: pulse rate/heart rate and blood pressure, respiratory rate, etc., including features of any of these. For example, the apparatuses and methods disclosed herein capture magnetic signals associated with a patient's physiological state, such as waveforms including pulse rate/heart rate, and/or blood pressure, through one or more magnetometers. In some cases, the magnetometers may be arranged in an array that may be worn by the patient. In some examples, the magnetometers may be arranged in a smartwatch form factor, although other form factors are contemplated (smart ring, bracelet, or the like).

Unlike conventional PPG or ECG-based systems that require optical contact or multi-limb electrode placement, the disclosed system enables non-contact or minimally contact-dependent biomagnetic sensing using a single wearable device, improving robustness to environmental and physiological variability.

The apparatus captures magnetic signals that can originate from the patient's wrist and be associated with the patient's pulse as detected at the wrist area. The patient can raise the apparatus (still being worn on the wrist) toward the patient's chest, particularly near a region of the heart. In this position, the apparatus can sense magnetic signals from the wrist and the heart simultaneously. The magnetic signals can be filtered to determine the patient's pulse at the heart and at the wrist. Using this pulse information, a pulse transit time (or other timing-based hemodynamic metrics) is determined. The timing-based hemodynamic metric may then be used to determine blood pressure.

Any of the methods described herein may be used to determine a patient's blood pressure using acoustically driven ferromagnetic resonance (ADFMR) sensors. The ADFMR sensors may be included within an apparatus. The apparatus may be a wearable device worn by the patient. The method may include determining, with one or more (ADFMR) sensors, a patient's pulse at a first location on the patient, determining with the ADFMR sensors, the patient's pulse at a second location, wherein the second location is different than the first location, calculating a pulse transit time based on the patient's pulse at the first location and the second location, and estimating the patient's blood pressure based on the calculated pulse transit time.

In any of the methods described herein the ADFMR sensors may be included in a wearable device worn about the patient's wrist. In some examples, the wearable device can have a form of a smart watch. Such a wearable device can operate in a first mode as a time piece and track and display time. The wearable device can operate in a second mode and capture magnetic signals and determine heart rate, pulse rate, and blood pressure. In some examples, determining the patient's pulse at the second location can include raising the patient's wearable device.

In any of the methods described herein, the ADFMR sensors may be included in a ring that can be worn about the patient's finger. In some other examples the ADFMR sensors can be included in a slim pouch. The pouch and the patient wearable device may each include wireless transceivers that allow data (including magnetic data) to be exchanged. In this manner, the pouch can act as a second set of ASFMR sensors. In some examples, the pouch can be placed in a pocket near the heart to enable continuous monitoring (e.g., heart rate and blood pressure monitoring).

Although many of the examples described herein are specific to a waveforms reflecting pulse rate and/or blood pressure monitoring, it should be understood that these methods and apparatuses described herein may be related to a variety of different metabolic and/or biological indicators, such as but not limited to pulse rate/heart rate, blood pressure, respiratory rate, etc. In particular, these methods and apparatuses may identify and/or isolate a waveform including any of these indicators and may analyze, track and/or monitor components of these waveforms, including (but not limited to) rate, as rate is just one of many metrics that can be derived from these signals.

In any of the methods described herein, determining the patient's pulse rate at the first location can include filtering out signals from the ADFMR sensors that are less than five hertz.

In any of the methods described herein, the second location can be on the chest adjacent to the heart. In any of the methods described herein, the first location can be the patient's wrist.

In any of the methods described herein, the pulse transit time can be the time that it takes blood to travel from a first site or location to a second site or location. In some examples, the pulse transit time can be the time it takes blood to travel from the heart to the patient's wrist.

Any of the methods described herein can also include continuously determining the patient's heart rate and blood pressure.

In any of the methods described herein, determining the patient's pulse at the first location can further include using physical information related blood vessels in the wrist to filter out noise and spurious signals from the ADFMR signals. In some examples, determining the patient's pulse at the second location can further include using physical information related to a heart muscle to filter out noise and spurious signal from the ADFMR signal.

In operation, the device may detect low-frequency MPG signals at the wrist and higher-frequency MCG signals when positioned near the chest. These signals may be separated by frequency filtering and processed to compute pulse transit time, or other timing-based hemodynamic metrics.

In any of the methods described herein, the ADFMR sensors can include an array of two or more ADFMR sensors.

For example, described herein are method of determining blood pressure of a patient using a sensing apparatus as described herein. These methods may include: detecting, with an array of magnetic sensors, first biomagnetic signals associated with blood flow at a first anatomical location and second biomagnetic signals associated with cardiac activity at a second anatomical location different from the first anatomical location; separating the first biomagnetic signals and the second biomagnetic signals based at least in part on frequency content; processing outputs of at least a first sensor and a second sensor using synthetic gradiometry comprising digitally weighting and subtracting signals from the second sensor from signals of the first sensor to reduce environmental noise; identifying temporal features in the first biomagnetic signals and the second biomagnetic signals; determining a pulse transit time based on a temporal difference between the identified temporal features; and estimating a blood pressure of the patient based on the pulse transit time.

Also described herein are apparatuses for performing any of these methods. For example, an apparatus may include: an array of magnetic sensors configured to detect biomagnetic signals associated with blood flow and cardiac activity; one or more processors; and a memory storing instructions that, when executed by the one or more processors, cause the apparatus to: separate biomagnetic signals corresponding to a first anatomical location and a second anatomical location based on frequency; apply synthetic gradiometry including digitally weighting and subtracting signals from at least one secondary sensor from a primary sensor; determine a pulse transit time based on temporally aligned features of the separated signals; and estimate a blood pressure of a patient based on the pulse transit time.

In some examples a system may include: a first sensor array configured to be positioned at a first anatomical location of a patient; a second sensor array configured to be positioned at a second anatomical location of the patient; and one or more processors configured to: receive biomagnetic signals from the first and second sensor arrays; process the biomagnetic signals using synthetic gradiometry; determine a pulse transit time between the first anatomical location and the second anatomical location; and estimate blood pressure based on the pulse transit time.

In any of these methods and apparatuses, the magnetic sensors may comprise an acoustically driven ferromagnetic resonance (ADFMR) sensors. The first biomagnetic signals may comprise magnetoplethysmography (MPG) signals and the second biomagnetic signals comprise magnetocardiography (MCG) signals.

In any of these methods and apparatuses, the signals from different anatomic locations may be filtered by different (adjacent and/or non-overlapping) frequency ranges. For example, separating may comprise filtering signals below approximately 5 Hz (e.g., below 4 Hz, 3 Hz, 2 Hz, 1 Hz, etc.) to isolate signals associated with the first anatomical location. For example, separating may comprise filtering signals between approximately 5 Hz and 50 Hz to isolate signals associated with the second anatomical location.

Digitally weighting may comprise adjusting a weighting factor to maximize a signal-to-noise ratio. Adjusting the weighting factor may comprises iterative optimization of spectral noise.

The synthetic gradiometry may be performed in a digital domain after analog-to-digital conversion. Weighting may be dynamically updated based on detected motion of the sensing apparatus. The array of magnetic sensors may simultaneously detect the first biomagnetic signals and the second biomagnetic signals using a single sensor array.

The sensing apparatus may be worn on a wrist and moved to a position proximate a heart of the patient to detect the second biomagnetic signals. Determining the pulse transit time may comprise identifying peaks in the first biomagnetic signals and the second biomagnetic signals. Determining the pulse transit time may comprises cross-correlating the first biomagnetic signals and the second biomagnetic signals. Estimating the blood pressure may comprise applying a multivariate model incorporating the pulse transit time and at least one additional physiological parameter.

The multivariate model may comprise a trained machine learning model.

In general, these methods and apparatuses may be particularly well suited for use with magnetic sensors comprising ADFMR sensors. The magnetic sensors may comprise magnetometers other than ADFMR sensors. Any of these apparatuses and methods may also include a motion sensor, wherein the processors are further configured to adjust weighting used in the synthetic gradiometry based on motion data.

In general, the apparatus may be configured as a wearable device, such as (but not limited to) a smartwatch, ring, etc.

The processors may be configured to apply band-pass filtering to separate biomagnetic signals. The processors may be configured to apply principal component analysis or independent component analysis to the biomagnetic signals. The processors may be configured to determine a signal quality metric and reject signals below a threshold.

The first sensor array and the second sensor array may communicate wirelessly. The second sensor array may be configured as a portable pouch positioned near a chest of the patient. The first sensor array may be configured to be worn on a wrist. At least one of the sensor arrays may comprise ADFMR sensors. The one or more processors may be configured to perform continuous monitoring of blood pressure. In general, determining the pulse transit time may comprise determining a hemodynamic timing metric correlated with blood pressure.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 shows an example of apparatus that includes a primary and a secondary Acoustically Driven Ferromagnetic Resonance (ADFMR) sensor.

FIG. 2 shows an example apparatus including four ADFMR sensors.

FIG. 3 shows an example wearable apparatus configured to measure pulse rate, heart rate, and blood pressure.

FIG. 4 shows a simplified diagram including an apparatus and a patient.

FIG. 5 is a flowchart showing an example method for determining a patient's blood pressure.

FIG. 6 shows graphs illustrating the relationship between estimated and real blood pressures.

FIG. 7 shows graphs illustrating the timing relationship between magnetic and optical blood flow signals.

### DETAILED DESCRIPTION

Acoustically Driven Ferromagnetic Resonance (ADFMR) sensors may be used to capture biomagnetic signals that are generated by the human body. In some examples ADFMR sensors may be used to determine a waveform including one or more of a patient's pulse rate, heart rate, and/or blood pressure and/or a metric assessed from such waveforms. The ADFMR sensors can sense biomagnetic signals related to blood flow at or near a patient's wrist and at or near a patient's heart. The ADFMR signals can be filtered and then used to determine a pulse rate or heart rate. These waveforms including pulse rate/heart rate can be used to determine the patient's blood pressure. In particular, the latency between heart beat timing may be used to derive blood pressure or other indicators.

In any of these methods and apparatuses synthetic gradiometry may be combined with single-array dual-location sensing to provide blood-flow related biometric information with a high degree of accuracy. Any of these methods and apparatuses may also incorporate frequency-based signal separation.

The use of ADFMR to detect waveforms including patient status information such as pulse rate/heart rate, and blood pressure can have several advantages over conventional techniques. For example, measurements based on ADFMR sensors do not rely on electrical or optical measurements which may be obfuscated by varying patient posture as well as the patient's skin condition such as sweat or humidity, or other anatomical differences, such as hair, skin color, or body composition, which can affect magnetic and/or optical signals. Magnetic (ADFMR) sensing can be much more consistent on a day-to-day basis and a wrist-worn device can force the patient to raise their arm to their chest to make the blood pressure measurement, resulting in a much more consistent posture between recordings. This may reduce the need for calibration and can provide stable recordings of blood pressure over time.

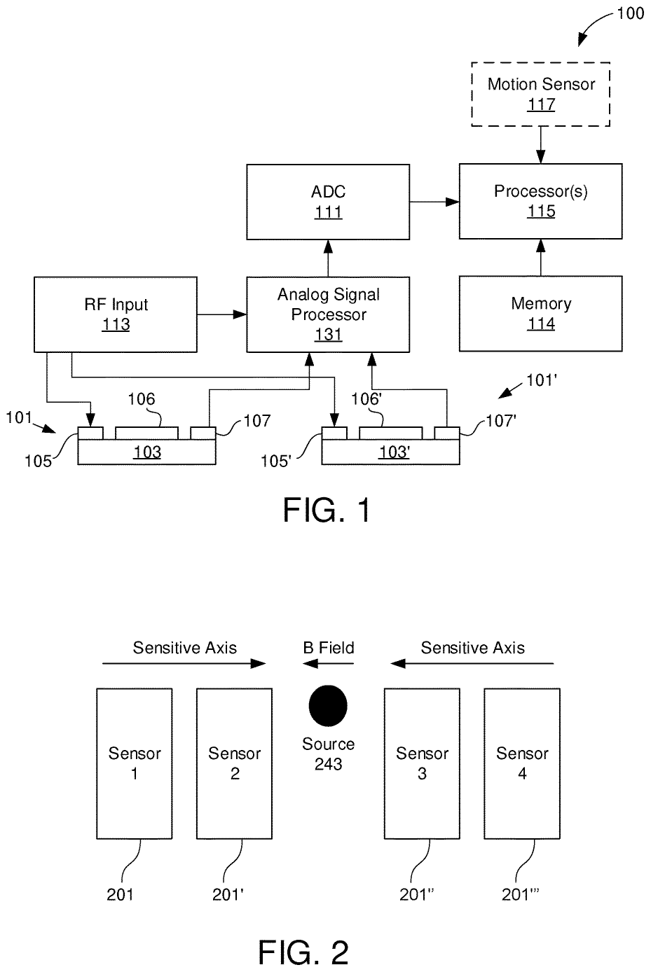

FIG. 1 shows an example of apparatus 100 that includes a primary and a secondary ADFMR sensor. The ADFMR sensors are configured to sense relatively small magnetic fields, such as those associated with biomagnetic signals. In some examples, synthetic gradiometry, as described herein, is used to refine the ADFMR signals. In the example of FIG. 1, two ADFMR sensors are shown for simplicity, however, more preferably additional (e.g., an array) of ADFMR sensors may be used.

In general, an ADFMR sensor, also referred to equivalently herein as an ADFMR magnetometer, is a device that uses acoustic waves, such as surface acoustic waves (SAWs), to drive ferromagnetic resonance in order to exploit the coupling between strain and magnetization in a magnetostrictive material. The apparatus 100 includes a first ADFMR sensor 101 and a second ADFMR sensor 101′. The first ADFMR sensor 101 includes an input transducer 105, an output acoustic transducer 107, a piezoelectric substrate 103, and a magnetostrictive material 106. In a similar manner, a second ADFMR sensor 101′ includes an input transducer 105′, an output acoustic transducer 107′, a piezoelectric substrate 103′ and a magnetostrictive material 106′.

The piezoelectric substrates 103 and 103′ can include any appropriate piezoelectric material, including but not limited to LiNbO3. The input acoustic transducers 105 and 105′ and the output acoustic transducers 107 and 107′ may be disposed on the same surface of the magnetostrictive material 106 and 106′. The acoustic transducers 105 and 105′ (e.g., an interdigitated transducer) are configured to activate the piezoelectric substrates (elements) 103 and 103′ to generate an acoustic wave. In any of these apparatuses, the input acoustic transducers 105 and 105′ may be interdigitated transducers with electrodes that are configured to excite surface acoustic waves (SAWs). The input acoustic transducers 105 and 105′ may be coupled (e.g., electrically coupled) to the source of input energy RF input 113 (a radio-frequency (RF) voltage source), to drive the application of acoustic energy through the device. The ADFMR sensors 101 and 101′ may each also include at least one output acoustic transducer 107, 107′ on the piezoelectric substrates 103 and 103′. The output acoustic transducers 107 and 107′ may be similar to the input acoustic transducers 105 and 105′. The output acoustic transducers 107 and 107′ may be electrically coupled to processing circuitry, e.g., readout circuitry, which are configured to detect a change in the acoustic wave to measure an unknown magnetic field to which the magnetostrictive material 106 and 106′ are exposed. In FIG. 1, the output of the output acoustic transducers 107 and 107′ are passed on to an (optional) analog signal processor 131 that may process the signals, the processing may include amplifying filtering, combining, etc. The apparatus 100 also includes an analog-to-digital converter (ADC) 111 to convert the sensed signals (passed from the analog signal processor 131) into the digital domain. One or more processor(s) 115 may receive the signals from the primary sensor 101, weigh the signals from the second sensor 101′, and subtract the weighted secondary signal from the primary sensor 101 to produce an output, which may be stored, further processed and/or displayed. The apparatus may optionally include one or more motion sensors 117 (e.g., an accelerometer and/or IMU), to adjust the weighting (calibration) frequency and/or for removing background environmental noise from the biosignals. The methods and apparatuses described herein may modify an ADFMR sensor to enhance the sensitivity of the sensor by including additional components (including circuitry) not shown in the simplified example of FIG. 1.

The apparatuses described herein may include hardware and/or software techniques to allow sensing low magnitude magnetic signals in ambient conditions. These methods and apparatuses may be used with one or more ADFMR sensors. For example, in any of these methods and apparatuses the sensors may be configured as an array of small (e.g., less than 30 mm², such as ~5×5 mm) ADFMR sensors that are configured to operate as a high density array providing optimized synthetic gradiometry as well as improved feature extraction and spatial filtering. Any of these methods and apparatuses may also or alternatively include ADFMR sensors having shielding specific to the ADFMR sensor, such as including small, light, and/or mobile shielding for each ADFMR sensor configured to attenuate environmental noise (e.g., ambient noise). This shielding may be applied to individual sensors, to groups of sensors within the array, and/or to the full array of sensors. In particular, any of the apparatuses described herein may include shielding encapsulating both the one or more sensors and the source of interest, including around the sensors and the portion of the body being sensed. For example, in a wrist-worn form factor (e.g., smart watch), the shielding may extend around the entire apparatus with the wrist inside it.

Any of these methods and apparatuses may also include signal processing for the output of the ADFMR sensor(s) to isolate signals (e.g., biosignals) from the background and maximize signal to noise ratio (SNR). Thus, the use of these methods and apparatuses may provide highly sensitive and reliable, and non-invasive detection of ambient signals. For example, these methods and apparatuses as described herein can be used to take ambient recordings of heart signals (e.g., magnetocardiography, MCG) using much lower sensitivity than previously shown to be feasible. These methods can also be used to record muscle activity (magnetomyography, MMG) in ambient conditions.

In some examples, any of the ADFMR sensors described herein can be high-density arrays of ADFMR sensors. These high density arrays allow optimized synthetic gradiometry as well as improved feature extraction and spatial filtering. Gradiometry may be used in combination with these ADFMR sensors to reduce or eliminate background noise in which the data from one or more secondary sensors (which may be further from the signal source) is subtracted from a primary sensor that is closer to the signal source. However, physical gradiometry, in which the outputs of the sensor are subtracted in analog, typically requires exact alignment of the primary and secondary sensors, and the noise floor is limited to the highest noise floor sensor of the pair. In contrast, synthetic gradiometry extends and improves upon physical gradiometry but cannot be easily employed in ambient conditions with most sensors due to their limited dynamic range.

Described herein are two variations of synthetic gradiometry that may be used with any of these methods and apparatuses including ADFMR sensors. These techniques may be used together or separately. The first technique, described in greater detail below, uses local shielding specific to the ADFMR sensor to reduce or eliminate ambient fields. Thes second technique uses signal processing to isolate biosignals from the ADFMR sensor output and maximize signal to noise ratio.

In general, these methods and apparatuses may include the use of multiple sensors, including a primary ADFMR sensor and one or more secondary ADFMR sensors, similar to physical gradiometry, but including further signal processing before combining/subtracting the signals. For example, a pair of sensors may be placed in proximity, much like in physical gradiometry, but the subtraction between the primary and secondary sensors may be applied after digitization. The weight of the signals between the primary and secondary sensors may be tuned in the digital domain prior to subtracting, including optimizing for signal to noise ratio (SNR). This tuning may be performed in an ongoing manner, in real time, and may include an analysis of the spectrum of the primary and/or secondary sensors and performing linear subtraction. Thus tuning may maximize the decrease in ambient signal and may minimize the ambient background. Any appropriate minimization technique may be used, including the use of an evolutionary algorithm, random walk, etc.

In some examples, the apparatus or method may include one or more sets of sensors that may be positioned to capture environmental noise in a region close to the subject along three orthogonal axes. This may allow characterization of the ongoing noise in real time, improving the discrimination of signal versus noise.

The tuning of the weights between the primary and secondary sensors in the digital domain may be performed in an ongoing manner, including recalibrating the relative weightings of the primary and secondary signals being subtracted with a predetermined frequency, or a variable (including adjustable) frequency. In some cases the frequency of calibration (“tuning”) may be adjusted based on environmental conditions, including movement/motion. Thus, in some cases these apparatuses may include a motion sensor (e.g., an accelerometer or inertial measurement unit, IMU), which may be used to adjust the rate of tuning. For example, the methods and apparatuses may be configured to calibrate the weights of the primary and secondary sensors at a rate that is based on the motion of the sensor(s) (e.g., this rate may be 1 Hz for light movement, and may be performed as higher rates when more movement is detected (e.g., >10 Hz). Thus, these methods and apparatuses may help correct for noise (artifact) due to or correlated with movement. The rate may be adjusted as part of the signal processing used for the direct gradiometry.

As mentioned, the signal (output) of the one or more secondary sensors that is/are subtracted from the primary sensor(s) may be weighted or scaled, in the digital domain, prior to subtracting. Thus, any of these methods and apparatuses may apply a weight to the signals from each secondary sensor that may be optimized to further lower noise. In some cases this weight may be determined using an optimization technique, e.g., optimization algorithm, such as a differential evolution algorithm, to minimize the spectral density outside a range of frequencies of interest. The weighting may account for differences in sensitivity between individual sensors as well as differences in noise amplitudes detected in each sensor due to the variable distances from the noise source. As mentioned, this tuning or calibration of the weights may be updated at a tuning frequency. In some cases the tuning frequency is least once every 100 ms (e.g., at 10 Hz or greater) to enable continuous movement of the sensors. The tuning frequency may be dynamic, as mentioned above.

Thus, correcting the output of an ADFMR sensor may generally include subtracting a weighted signal/output based on the output of one or more secondary ADFMR sensors from the primary ADFMR sensor signal. A maximization algorithm may be used to see the weighting values. All of these steps may be performed in the digital domain.

For example, any of these methods, or apparatuses configured to perform them, may comprise synthetic gradiometry including calibrating (e.g. weighting) the signal to be subtracted from one or more secondary sensors. The calibration weights may initially be set to 1 (e.g., equally weighted). In some cases the spectral densities of the secondary sensor(s) and the primary sensor(s) may be calculated. The level of noise present in the signals may be determined; in some examples the noise level may be determined by integrating over the frequencies outside of the range of interest (e.g., one or more regions of frequencies not of interest) and that may be common for noise sources. Alternatively or additionally, the SNR may be estimated by dividing the amplitude of a known signal frequency (or frequency band) by the integrated noise. Other metrics can also be utilized.

The calibration weights may be updated to minimize this estimated level of noise and/or maximize the SNR. For example the calibration weight (“weights”) may be adjusted/updated using either gradient-based or stochastic methods that minimize the calculated level of noise, maximize the SNR, or any other technique appropriate for the selected optimization metric. In some cases a gradient based method may be used to calculate the gradient (derivative) used, whereas a stochastic method may generate random variables to search a space.

This process may be repeated until the noise level is deemed low enough (e.g., it is less than and/or equal to a threshold noise level). Alternatively or additionally, this process may be repeated until the noise level does not change more than a predetermined value between iterations, and/or until a set number of iterations have been performed.

Once this optimized weight level has been estimated as described above, the calibration weight for the secondary sensor outputs (signals) may be applied, and the weighted output subtracted from the primary sensor output. The calibration weights may be recalculated as described above at the weighting frequency, e.g., after waiting for the period of time determined by the weighting frequency, such as 100 ms as mentioned above. In some cases recalibration (adjusting the calibration weight) may be performed more frequently, and/or continuously, or less frequently (e.g., 1 Hz), as mentioned.

The methods and apparatuses described herein are particularly useful in allowing a flexible form factor that may be adjustably or movably applied to a body (e.g., worn). Any of the apparatuses described herein may be configured as part of a wearable garment or accessory and may permit adjustable/flexible positioning of the various sensors, including relative changes in the positions between primary and secondary ADFMR sensors. This is particularly surprising in comparison to other magnetic sensing systems or other sensing systems in general that reduce noise by attempting to subtract from a secondary sensor, which is widely thought that the primary and secondary sensors must be in fixed positions relative to each other. Thus, in general, the individual sensors (e.g., primary and secondary sensors) may be movably or flexibly connected to each other and to the other components of the apparatus (e.g., RF input, circuitry, etc.). The use of tuning, including real-time tuning and/or adaptive tuning, as described herein may permit this flexibility without significant loss of sensitivity and accuracy.

Although the example shown in FIG. 1 includes a single secondary sensor 101′, in some cases three or more (e.g., four) secondary sensors may be used, arranged in the different directions/axes. In any of these apparatuses and methods, synthetic gradiometry does not have to pre-define ‘signal’ and ‘reference’ sensors. For example any of these apparatuses may include an array of N sensors and may perform subtraction operations among them. The apparatus or method may update those relationships dynamically. For example, for 4 sensors (e.g., indicated by A, B, C, D), the method or apparatus may dynamically have an output of E=A−B, F=C−D, or E=A−C, F=B−D, or E=A−B, F=C−E, G=D−E, etc. depending on dynamic positioning, noise, motion, etc.

The apparatus 100 may include a memory 114 coupled to the processor(s) 115. The memory 114 can include a non-transitory computer-readable storage medium (e.g., one or more nonvolatile memory elements, such as EPROM, EEPROM, Flash memory, a hard drive, etc.) that may store software modules. Each software modules can include program instructions that, when executed by the processor(s) 115, may cause the apparatus 100 to perform one or more functions. Thus, the non-transitory computer-readable storage medium of memory 114 may include instructions for performing all or a portion of the operations described herein.

Multiple sets of sensors may be used for gradiometry according to the techniques described herein. FIG. 2 schematically illustrates an example including four sensors 201, 201′, 201″, 201′″ that may be used as pairs for sensing the magnetic field from a source 243 emitting a magnetic field (B field). In this example, a single gradiometry configuration is shown by the configuration in which the output of sensor 2 is subtracted from the scaled/weighted output of sensor 1 (“Gradiometry1”). Similarly a second single gradiometry configuration is also shown, in which the output of sensor 3 is subtracted from the scaled/weighted output of sensor 4 (“Gradiometry2”). This example also illustrates a “double gradiometry”) configuration, in which the resulting outputs are combined, e.g., Gradiometry 1+Gradiometry2.

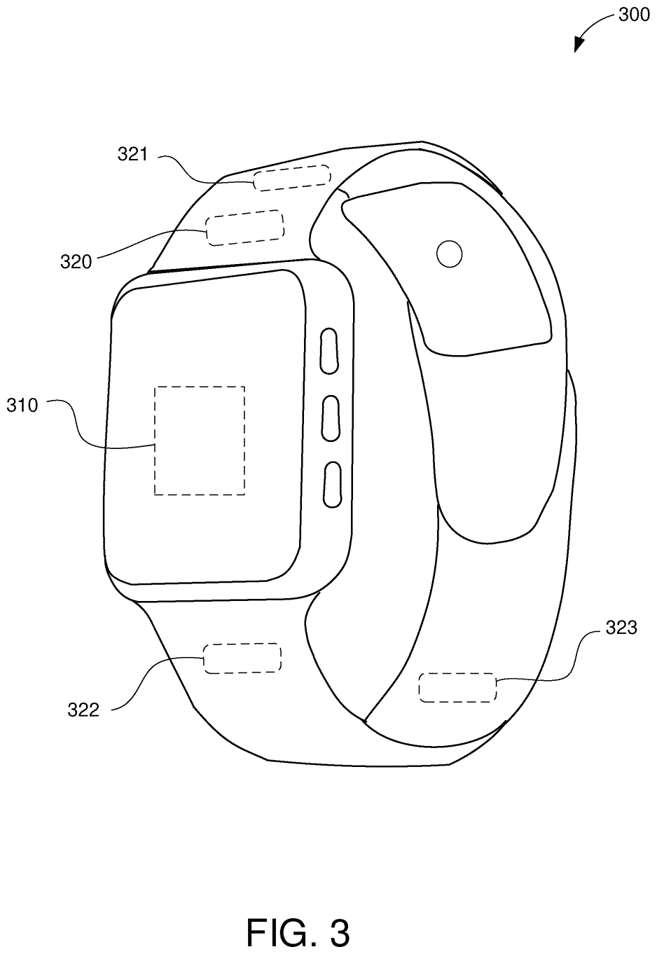

FIG. 3 shows an example wearable apparatus 300 configured to measure pulse rate, heart rate, and blood pressure. As shown, the apparatus 300 can take the form of a smart watch, however of forms are contemplated. For example, the apparatus 300 may take the form of a ring (that may be worn around a finger or thumb), a bracelet, or the like. The apparatus 300 can include a processing unit 310 and ADFMR sensors 320, 321, 322, and 323. Although only four ADFMR sensors 320-323 are shown, in other examples, the apparatus may include any feasible number of ADFMR sensors.

The ADFMR sensors 320-323 are configured to measure magnetic signals, in particular, magnetic signals produced by or associated with biologic functions such as blood flow. In some examples, when the apparatus 300 is worn on a patient's wrist, the ADFMR sensors 320-323 can receive magnetoplethysmography (MPG) signals. Notably, MPG signals are typically less than five hertz (<5 Hz). MPG signals may vary according to blood flow near the apparatus 300. If the apparatus 300 is worn on the patient's wrist, then the MPG signals may be related to blood flow through the patient's radial artery. In some examples, the MPG signals can be used to determine the patient's pulse rate. In any of these methods and apparatuses, the same ADFMR sensors may be used to detect other biological waveforms, including MMG signals. For example, these ADFMR sensors may be used to simultaneously measure MMG signals and MPG signals or other signals having non-overlapping frequency bands.

The ADFMR sensors 320-323 are coupled to the processing unit 310. The processing unit can include some or all of the components included in the apparatus 100 of FIG. 1. For example, the processing unit 310 may include the RF input 113, the audio signal processor 131, the memory 114, the ADC 111, and the processor(s) 115. Thus, the processors(s) 115 can process magnetic signals received by the ADFMR sensors 320-323 using gradiometry techniques described herein to determine blood flow and related pulse rate through MPG signals at or near the patient's wrist.

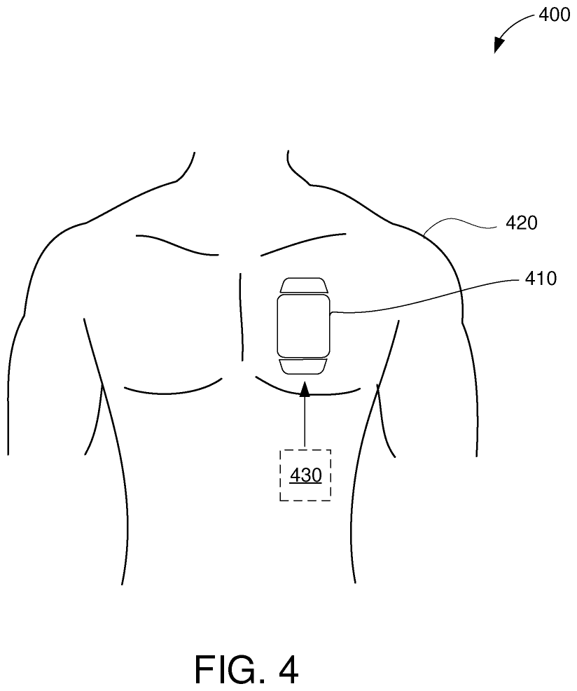

In some examples, the patient can move the apparatus 300 to be near the patient's heart while still being worn on the wrist. For example, patient can raise his/her wrist while wearing the apparatus 300 to a position near the patient's heart. In this position, the apparatus 300 can simultaneously capture magnetocardiography (MCG) signals associated with blood flow in and near the heart as well as the MPG signals described above. FIG. 4 shows a simplified diagram 400 including an apparatus 410 and a patient 420. The apparatus 410, which is an example of the apparatus 100 of FIG. 1 or the apparatus 300 of FIG. 3, may be held over the heart of the patient 420.

In some examples, the MCG signals may be between 1-100 Hz (e.g., between 5-50 Hz, between 4-12 Hz, between 1-75 Hz, between 5-75 Hz, etc.). Thus, the processing unit 310, through low-pass and/or band-pass filtering, can isolate and detect MPG and MCG signals. In some examples, the filtering may be performed in the digital domain by the processor(s) 115. In some examples, the filtering can be any bandwidth filtering.

When the patient brings their wrist to their chest, the apparatus 300 can capture and/or record both MPG and MCG simultaneously with the same array of sensors. The apparatus 300 can determine a pulse transit time (PTT) of blood flow from the patient's heart to the patient's wrist (using MCG and MPG data) to determine and/or estimate the patient's systolic and diastolic blood pressure. Advantageously, the measurements (MPG, MCG, PTT and blood pressure) may be much more consistent due to the nature of the body being invisible to magnetic signals as well as an enforced consistency in posture during recordings. Thus, the using the apparatus 300 to determine blood pressure in this manner may reduce confounding factors affecting measurement accuracy and consistency. In addition, the apparatus 300 simply requires a single set of magnetometers (ADFMR sensors) on one limb as opposed to conventional methods that use both photoplethysmography (PPG) and electrocardiogram (ECG) sensors requiring both limbs.

In some examples, continuous measurement may be desired. For example, a patient may have a chronic condition that can benefit from continuous pulse, heart, or blood pressure monitoring. In those cases, an optional second array of ADFMR sensors 430 (shown optional with dashed lines) may be placed in a shirt pocket or similar location. The second array of ADFMR sensors 430 may include a wireless transceiver (wi-fi, Bluetooth, or the like) to enable wireless communication with the apparatus 410. Then, using information from the apparatus 410 and the second array of ADFMR sensors 430, the apparatus 410 can continuously monitor the patient's heart rate, pulse rate, and/or blood pressure. The second array of ADFMR sensors 430 may be included in a slim pouch or packet that comfortably fits within a shirt pocket or can be worn from a lanyard under the patient's clothing.

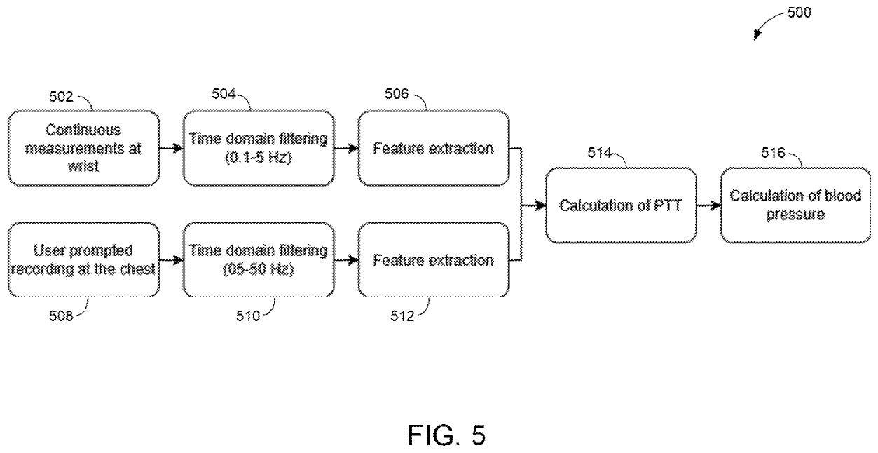

FIG. 5 is a flowchart showing an example method 500 for determining a patient's blood pressure. Some examples may perform the operations described herein with additional operations, fewer operations, operations in a different order, operations in parallel, and some operations differently. The method 500 is described below with respect to the apparatus 300 of FIG. 3, however, the method 500 may be performed by any other suitable system or device.

The method 500 begins in block 502 as the apparatus 300 makes continuous magnetic measurements. In some examples, the apparatus 300 may be worn on the patient's wrist. Thus, the apparatus 300 may make continuous magnetic measurements regarding blood flow near or around the patient's wrist. In some examples, the apparatus 300 may receive MPG signals to determine blood flow information near and around the patient's wrist, including blood flow information associated with the patient's radial artery.

Next, in block 504 the MPG signals may be filtered. In some examples, the MPG signals may be time domain filtered to remove power line noise (e.g., 50 Hz or 60 Hz noise). Alternately, or additionally, the MPG signal may be bandwidth filtered to allow only signals between 0.1 Hz and 5 Hz. MPG signals (signals associated with wrist blood flow) typically are between 0.1 Hz and 5 Hz.

Next, in block 506 the apparatus 300 performs feature extraction. Feature extraction processes can refine the time domain filtered MPG signal data so that blood flow information is more readily determined. In some examples, feature extraction processing can include spatial filtering. Spatial filtering can advantageously use physical information related to the magnetic signal source to filter out noise and spurious signals. For example, wrist related magnetic signals coming from the blood vessels in the wrist are quite small and therefore generate a more localized signal. In some other examples, principal component analysis (PCA), independent component analysis (ICA), and/or synthetic gradiometry may be used to extract blood flow features information from the MPG signals. In some examples, a peak detection algorithm may be used to determine blood flow information. Alternatively or additionally, other features may be detected, including, for example, maximum slope (i.e., peak velocity), to identify the timing of different features of the waveforms.

In block 508, the patient (user) is prompted to measure heart-related blood flow information. For example, the patient may be prompted to hold the apparatus 300 near his/her heart. In this position, the apparatus 300 can collect MCG signals from patient's heart as well as MPG signals from the patient's wrist. In some examples, the MPG signals may be processed as described with respect to blocks 502, 504, and 506.

Next, in block 510 the MPG signals are time domain filtered. In some examples, MPG signals are filtered to remove power line noise (e.g., 50 Hz or 60 Hz noise). Alternately, or additionally, the MPG signal may be bandwidth filtered to allow only signals between 5 and 50 Hz. MCG signals (signals associated with wrist blood flow in and around the heart) typically are between 5 Hz and 50 Hz).

Next, in block 512 the apparatus 300 performs feature extraction, similar to block 506. For example, feature extraction processes can refine the time domain filtered MCG signal data so that blood flow information is more readily determined. In some examples, feature extraction processing can include spatial filtering. Spatial filtering can advantageously use physical information related to the magnetic signal source to filter out noise and spurious signals. For example, since the heart is a large muscle, the magnetic field gradient falls off more slowly and generates are more spatially distributed field, compared to wrist related magnetic fields. This characteristic may be used for feature extraction. In some other examples, principal component analysis (PCA), independent component analysis (ICA), and/or synthetic gradiometry may be used to extract blood flow features information from the MCG signals. In some examples, a peak detection algorithm may be used to determine blood flow information. In some examples, at least ten pulses (peaks) are determined to lower variability on timing.

Next, in block 514 the apparatus 300 calculates the PTT based on the MPG signals (from block 506) and MCG signals (from block 512). In some examples, the PTT is calculated based on the time of a detected MPG signal peak and the time of a detected MCG signal peak (i.e., time of MPG signal peak-time of MCG signal peak).

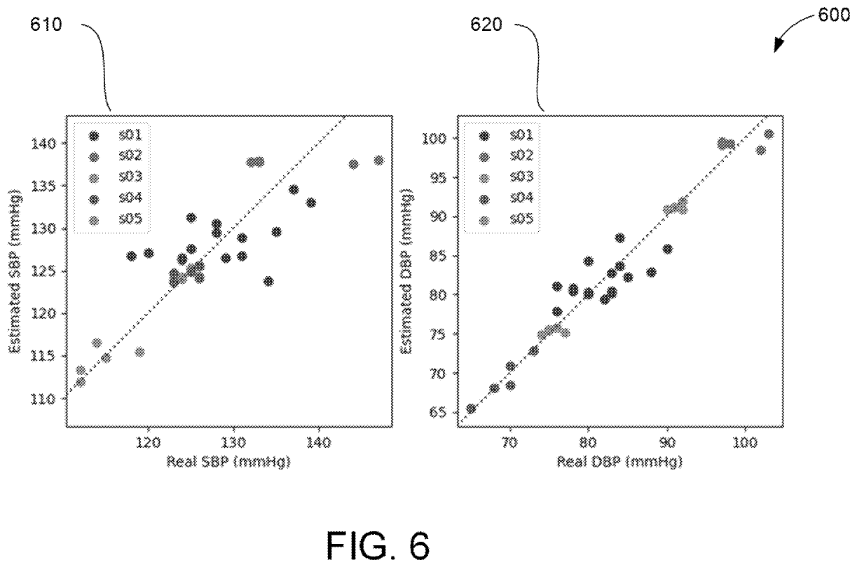

Next, in block 516 the apparatus 300 calculates the patient's blood pressure based on the calculated PTT. For example, the patient's blood pressure (BP) may be calculated as

$$BP = \frac{{K}_{1}}{PTT} + {K}_{2} \qquad ( equation 1 )$$

Where K₁ and K₂ are constants. In some examples, the constants can be static and used across different patients or they may be determined through a calibration procedure for each patient for higher accuracy. For example, K₁ and K₂ may be estimated empirically (e.g., fitting multiple measurements); as shown in FIG. 6, a scatter plot of varying PTT and measured BP may be measured from a traditional method (e.g., using a blood pressure cuff) and used to generate the constants that best fit the correlation between the two. In some examples, the static variables (K1 and K2) that may be used across patients may be determined by obtaining simultaneous PTT data from ADFMR sensors and blood pressure data (e.g., obtained from a traditional method, such as blood pressure cuff) from a representative population sample, including variation in age, gender, body type, etc. The data may be fit to a function so that the prediction is accurate and robust. Patient demographic information may be used to improve the selection of constants for the individual. In some cases, even more accurate results may be achieved for an individual using a calibration procedure that may include recording data with a BP cuff on a specific patient. In some cases the apparatus or method may be configured to track changes in BP rather than absolute values (e.g., to know if their BP is trending above or below a personal average). In this case, a calibration process might not be used.

FIG. 6 shows graphs 600 illustrating the relationship between estimated and real blood pressures. The estimated blood pressure may be determined as described herein using magnetic fields and ADFMR sensors to determine PTT. The real blood pressure may be determined using any conventional methods and apparatuses. Graph 610 compares real systolic blood pressure (on the x-axis) to an estimated systolic blood pressure (on the y-axis). Graph 610 shows a good linear correlation between real and estimated systolic blood pressures. Graph 620 compares real diastolic blood pressure (on the x-axis) to an estimated diastolic blood pressure (on the y-axis). Graph 620 shows a good linear correlation between real and estimated diastolic blood pressures.

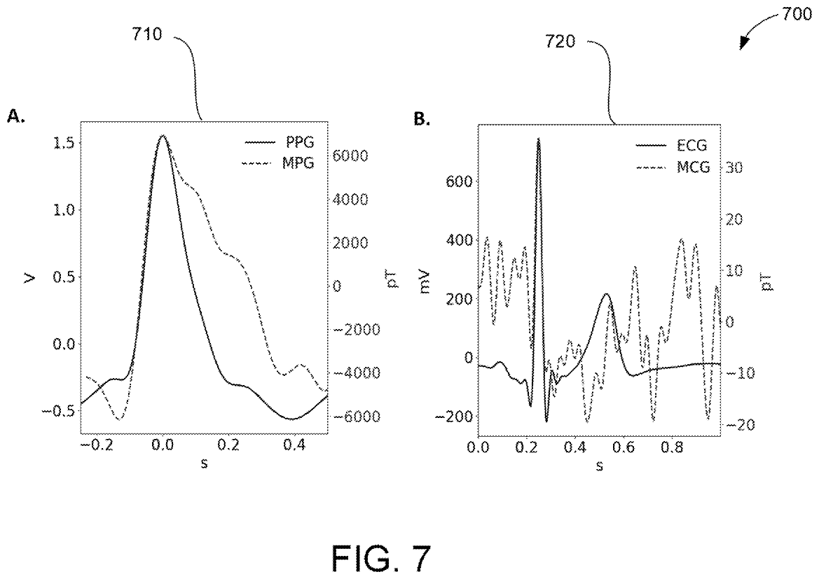

FIG. 7 shows graphs 700 illustrating the timing relationship between magnetic and optical blood flow signals. Graph 710 shows the timing relationship between photoplethysmography-based (PPG) and magnetoplethysmography (MPG) heart signals. In a similar manner, graph 720 shows the timing relationship between electrocardiography (ECG) and magnetocardiography (MCG) heart signals. Graph 720 particularly shows similar timing in the QRS complex. Because of the timing similarities shown in PPG-MPG signals (graph 710) and ECG-MCG signals (graph 720), magnetic signal-based pulse rate, heart rate, and blood pressure methods and apparatuses may be used in place of other methods and apparatuses. Similar data may show the relationship between magnetic versus electrical heart signals, which may be reflected in the PPG signal.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of determining and monitoring blood pressure of a patient with a wearable device, the method comprising:
determining, with acoustically driven ferromagnetic resonance (ADFMR) sensors, a patient's pulse at a first location on the patient;
determining, with the ADFMR sensors, the patient's pulse at a second location on the patient, wherein the second location is different than the first location;
calculating a pulse transit time based on the patient's pulse at the first location and the second location; and
estimating the patient's blood pressure based on the calculated pulse transit time.

2. The method of claim 1, wherein the ADFMR sensors are included in a wearable device worn around the patient's wrist.

3. The method of claim 2, wherein determining the patient's pulse at the second location includes raising the patient's wearable device to a region near the patient's heart.

4. The method of claim 1, wherein the ADFMR sensors are included in a ring worn about the patient's finger.

5. The method of claim 1, wherein the ADFMR sensors are included in a slim pouch.

6. The method of claim 1, wherein determining the patient's pulse rate at the first location includes bandwidth filtering signals from the ADFMR sensors to less than five hertz.

7. The method of claim 1, wherein the second location is on the patient's chest adjacent to the patient's heart.

8. The method of claim 1, wherein the first location is the patient's wrist.

9. The method of claim 1, wherein the pulse transit time is the time for blood to travel from a first site to a second site.

10. The method of claim 1, further comprising continuously determining the patient's heart rate and blood pressure.

11. The method of claim 1, wherein the ADFMR sensors include an array of two or more ADFMR sensors.

12. An apparatus comprising:
an array of magnetic sensors configured to detect biomagnetic signals associated with blood flow and cardiac activity;
one or more processors; and
a memory storing instructions that, when executed by the one or more processors, cause the apparatus to:
separate biomagnetic signals corresponding to a first anatomical location and a second anatomical location based on frequency;
apply synthetic gradiometry including digitally weighting and subtracting signals from at least one secondary sensor from a primary sensor;
determine a pulse transit time based on temporally aligned features of the separated signals; and
estimate a blood pressure of a patient based on the pulse transit time.

13. The apparatus of claim 12, wherein the magnetic sensors comprise ADFMR sensors.

14. The apparatus of claim 12, further comprising a motion sensor, wherein the processors are further configured to adjust weighting used in the synthetic gradiometry based on motion data.

15. The apparatus of claim 12, wherein the apparatus is configured as a wearable device.

16. The apparatus of claim 15, wherein the wearable device comprises a smartwatch.

17. The apparatus of claim 15, wherein the wearable device comprises a ring.

18. The apparatus of claim 12, wherein the processors are further configured to apply band-pass filtering to separate biomagnetic signals.

19. The apparatus of claim 12, wherein the processors are configured to apply principal component analysis or independent component analysis to the biomagnetic signals.

20. The apparatus of claim 12, wherein the processors are configured to determine a signal quality metric and reject signals below a threshold.
